SEQUENCE LISTING (21 sequences)

SEQ ID NO: 1 - protein, 11 aa, synthetic construct
  REGION 1..11: note = synthetic peptide
QQRFEWEFEQQ

SEQ ID NO: 2 - protein, 11 aa, synthetic construct
  REGION 1..11: note = synthetic peptide
  MOD_RES 5: note = Orn
  MOD_RES 7: note = Orn
QQRFXWXFEQQ

SEQ ID NO: 3 - protein, 11 aa, synthetic construct
  REGION 1..11: note = synthetc peptide
SSRFEWEFESS

SEQ ID NO: 4 - protein, 11 aa, synthetic construct
  REGION 1..11: note = synthetic peptide
  MOD_RES 5: note = Orn
  MOD_RES 7: note = Orn
SSRFXWXFESS

SEQ ID NO: 5 - protein, 11 aa, synthetic construct
  REGION 1..11: note = synthetic peptide
EQEFEWEFEQE

SEQ ID NO: 6 - protein, 11 aa, synthetic construct
  REGION 1..11: note = synthetic peptide
  MOD_RES 3: note = Orn
  MOD_RES 5: note = Orn
  MOD_RES 7: note = Orn
  MOD_RES 9: note = Orn
QQXFXWXFXQQ

SEQ ID NO: 7 - protein, 11 aa, synthetic construct
  REGION 1..11: note = synthetic peptide
NNRFEWEFENN

SEQ ID NO: 8 - protein, 11 aa, synthetic construct
  REGION 1..11: note = synthetic peptide
  MOD_RES 5: note = Orn
  MOD_RES 7: note = Orn
NNRFXWXFENN

SEQ ID NO: 9 - protein, 11 aa, synthetic construct
  REGION 1..11: note = synthtic peptide
TTRFEWEFETT

SEQ ID NO: 10 - protein, 11 aa, synthetic construct
  REGION 1..11: note = synthetic peptide
  MOD_RES 5: note = Orn
  MOD_RES 7: note = Orn
TTRFXWXFETT

SEQ ID NO: 11 - protein, 11 aa, synthetic construct
  REGION 1..11: note = synthetic peptide
  MOD_RES 5: note = Orn
  MOD_RES 7: note = Orn
QQRQXQXQEQQ

SEQ ID NO: 12 - protein, 11 aa, synthetic construct
  REGION 1..11: note = synthetic peptide
QQRQEQEQEQQ

SEQ ID NO: 13 - protein, 11 aa, synthetic construct
  REGION 1..11: note = synthetic peptide
SSRQEQEQESS

SEQ ID NO: 14 - protein, 11 aa, synthetic construct
  REGION 1..11: note = synthetic peptide
SSRSESESESS

SEQ ID NO: 15 - protein, 11 aa, synthetic construct
  REGION 1..11: note = synthetic peptide
  MOD_RES 3: note = Orn
  MOD_RES 5: note = Orn
  MOD_RES 7: note = Orn
  MOD_RES 9: note = Orn
QQXQXQXQXQQ

SEQ ID NO: 16 - protein, 11 aa, synthetic construct
  REGION 1..11: note = synthetic peptide
EQEQEQEQEQE

SEQ ID NO: 17 - protein, 11 aa, synthetic construct
  REGION 1..11: note = synthetic peptide
  MOD_RES 1: note = Orn
  MOD_RES 3: note = Orn
  MOD_RES 5: note = Orn
  MOD_RES 7: note = Orn
  MOD_RES 9: note = Orn
  MOD_RES 11: note = Orn
XQXFXWXFXQX

SEQ ID NO: 18 - protein, 11 aa, synthetic construct
  REGION 1..11: note = synthetic peptide
QQEFEWEFEQQ

SEQ ID NO: 19 - protein, 11 aa, synthetic construct
  REGION 1..11: note = synthetic peptide
ESEFEWEFESE

SEQ ID NO: 20 - protein, 11 aa, synthetic construct
  REGION 1..11: note = synthetic peptide
  MOD_RES 3: note = Orn
  MOD_RES 5: note = Orn
  MOD_RES 7: note = Orn
  MOD_RES 9: note = Orn
SSXFXWXFXSS

SEQ ID NO: 21 - protein, 11 aa, synthetic construct
  REGION 1..11: note = synthetic peptide
  MOD_RES 1: note = Orn
  MOD_RES 3: note = Orn
  MOD_RES 5: note = Orn
  MOD_RES 7: note = Orn
  MOD_RES 9: note = Orn
  MOD_RES 11: note = Orn
XSXFXWXFXSX

(19) United States
(12) Patent Application Publication
(10) Pub. No.: US 2025/0043142 A1
(43) Pub. Date: Feb. 6, 2025

(54) IN SITU MINERALIZATION OF 3D PRINTED METASTABLE CALCIUM SPECIES

(71) Applicant: Omya International AG, Oftringen (CH)

(72) Inventors: Gabriela MELO RODRIGUEZ, Aarau (CH); Joachim SCHOELKOPF, Oberkulm (CH); Lucy KIND, Staufen (DE); Rosario Maurizio GULLO, Boniswil (CH)

(73) Assignee: Omya International AG, Oftringen (CH)

(21) Appl. No.: 18/715,016

(22) PCT Filed: Nov. 28, 2022
(86) PCT No.: PCT/EP2022/083515

§ 371 Date: May 30, 2024

(30) Foreign Application Priority Data
Dec. 2, 2021 (EP) 21212027.3

(51) Int. Cl. C09D 101/28; A61C 8/00; A61K 6/17; A61K 6/71; A61K 6/74; A61K 6/898; A61L 27/20; A61L 27/22; A61L 27/44; A61L 27/52; B29C 64/118; B29C 64/30; B29K 1/00; B29K 509/02; B29L 31/00; B33Y 40/00; B33Y 40/20; B33Y 70/00; B33Y 80/00; C09D 7/40; C09D 7/61; C09D 7/63

(52) U.S. Cl. CPC C09D 101/28; A61C 8/0012; A61K 6/17; A61K 6/71; A61K 6/74; A61K 6/898; A61L 27/20; A61L 27/227; A61L 27/446; A61L 27/52; B29C 64/118; B29C 64/30; B33Y 40/00; B33Y 40/20; B33Y 70/00; B33Y 80/00; C09D 7/61; C09D 7/63; C09D 7/67; C09D 7/68; A61L 2430/02; B29K 2001/08; B29K 2089/00; B29K 2509/02; B29L 2031/7532

(57) ABSTRACT

The present invention refers to a biomimetic minerizable 3D-printing ink, a method for the production of such a biomimetic minerizable 3D-printing ink, a method for the production of a biomineralized 3D-printed article, a biomineralized 3D-printed article as well as the use of a crystallization trigger which is an oligopeptide selected from the group comprising an oligopeptide of the HABP family and an oligopeptide of the P11-family for 3D printing.

The present invention refers to a biomimetic minerizable 3D-printing ink, a method for the production of such a biomimetic minerizable 3D-printing ink, a method for the production of a biomineralized 3D-printed article, a biomineralized 3D-printed article as well as the use of a crystallization trigger which is an oligopeptide selected from the group comprising an oligopeptide of the HABP family and an oligopeptide of the P11-family for 3D printing.

3D printing is typically carried out in that a material such as plastics or liquids is deposited, joined or solidified under computer control to create a three-dimensional object, whereby the object is typically build up by forming one layer after the other layer. In view of the wide variety of objects that can be build up by this technology in a customized manner, 3D printing can be used in different application fields. Also, in the field of biomaterials and tissue engineering, this technology is of major interest for the customized preparation of dental reconstruction material, ceramic substitute, mollusk shell substitute, nacre substitute, dentin substitute, tooth substitute or bone substitute.

For example, CN108126244 A is directed to a biomineralized 3D printing ink characterized by comprising a hydrogel; the hydrogel includes a concentrated sodium alginate and sodium alginate complex containing a phosphorus source; the phosphorus source concentration range is 0.1 mol/L 1.5 mol/L; the mass fraction of sodium alginate in the hydrogel is 10% to 20%; and the mass fraction of alginate complex in the hydrogel is 5% to 20%. CN106668934 B refers to a preparation method of a calcium phosphate-based 3D printing material for biomedicine, which comprises immersing amorphous calcium phosphate in pure water containing metal ions, rare earth ions and water-soluble polymers, and drying and grinding to obtain a calcium phosphate-based 3D printing material, the water-soluble polymer is at least one of polyvinyl alcohol, sodium carboxymethyl cellulose, hydroxyethyl cellulose, polyvinyl pyrrolidone, and starch. CN109999223 A refers to a regulatable artificial bone repair system for repairing alveolar bone, which is characterized by the following components: 1 degradable natural polymer, 2 degradable artificial polymer, 3 bioceramic, 4 metal. CN108295306 A refers to a three-dimensional printing hydrogel material comprising a mesoporous nano calcium phosphate particle filler, comprising: calcium phosphate particles and a hydrogel material, the calcium phosphate particles being uniformly dispersed in a hydrogel material, the calcium phosphate; the surface of the particles is modified with polyethylene glycol, the calcium phosphate particles are hollow spheres or ellipsoids with a crystalline outer shell, and the pore size of the calcium phosphate particles is mainly between 20 nm and 70 nm. US2018243980 A1 refers to a system for three-dimensional printing and mineralizing a polymer, the system comprising: a three-dimensional printer unit with a syringe extruder; a fluid delivery system operatively coupled to the three-dimensional printer unit; and a control unit operatively coupled to the three-dimensional printer unit and fluid delivery system, the control unit configured to: cause the three-dimensional printer unit to print a portion of a three-dimensional polymer object; cause the fluid delivery system to flush the portion of the three-dimensional polymer object with a fluid to mineralize the portion of the three-dimensional polymer object; and cause the three-dimensional printer unit to print a subsequent portion of the three-dimensional polymer object.

CN104147641 B refers to a bone repair material for personalization, including organic and inorganic materials with a mass ratio of 0.1-0.6:1, and a lipid microsphere powder coated with protein; wherein the organic material is selected from the group consisting of PLGA, PLA and PGA, wherein the PLA: PGλ=5%-95%: 95%-5%, the molecular weight range is 8000-250000; the PLA molecular weight ranges from 8000-250000; The PGA molecular weight range is 8000-250000; the inorganic material is a calcium phosphate salt selected from one or more of β-tricalcium phosphate, α-tricalcium phosphate, hydroxyapatite and tetracalcium phosphate; the protein encapsulated in the lipid microspheres is recombinant human interferon beta, and the content of recombinant human interferon beta in each lipid microsphere powder is 50-200 ng/serving. KR20180128227 A refers to a filament composite resin composition for FDM-3D printing, comprising biocompatible polymers; bone activated ceramics; and a dispersant, wherein the bone-active ceramic has a particle diameter of 100 nm to 800 nm. CN108815574 A refers to a bone repair hydrogel stent, characterized in that the bone repair hydrogel stent comprises a polymer gel carrier and mineralized nano bone particles, the polymer gel carrier comprising a polymer gel and a matrix metalloproteinase In response to the peptide chain, the polymer gel is directly cross-linked with the matrix metalloproteinase-responsive peptide chain, and the bone-repaired hydrogel scaffold is subjected to 3D printing under ultraviolet light excitation.

However, 3D printed articles of the prior art suffer from several disadvantages, especially calcium based 3D printed articles that may be used to mimic bone, teeth, shell and/or nacre must be typically sintered in order to achieve sufficient hardness and crystallization.

Thus, 3D printed objects such as calcium based 3D printed objects that may be used to mimic bone, teeth, shell and/or nacre can be prepared, but typically, these objects must be sintered in order to achieve sufficient hardness and crystallization.

Thus, it is the object of the present invention to provide a 3D-printing ink that is suitable for the preparation of biomineralized 3D-printed articles based on calcium materials. A further object of the present invention is that the biomineralized 3D-printed article is suitable to mimic bone, teeth, shell and/or nacre. Another object of the present invention is that the biomimetic mineralized 3D-printed article provides sufficient hardness and that the hardening can be achieved without sintering.

One or more of the foregoing and other objects are solved by the subject-matter as defined herein in the independent claim. Advantageous embodiments of the present invention are defined in the corresponding sub-claims.

The present invention thus relates to a biomimetic minerizable 3D-printing ink comprising
a) a calcium cation-based compound being metastable calcium carbonate or metastable calcium phosphate,
b) a carrier material, and
c) a crystallization trigger.

According to one embodiment, the calcium cation-based compound has a crystallinity of less than 50 wt.-%, preferably of less than 40 wt.-%, more preferably of less than 30 wt.-% and most preferably of less than 20 wt.-%, based on the total weight of the calcium cation-based compound.

According to another embodiment, the calcium cation-based compound has a weight median particle size d₅₀ as determined by by dynamic light scattering in the range from 1 to 500 nm, preferably from 50 to 400 nm and most preferably from 100 to 350 nm.

According to yet another embodiment, the carrier material is a material suitable to form a hydrogel, preferably the carrier material is selected from the group comprising gelatin, methylcellulose, alginate, agarose, fibrin, hyaluronic acid, proteins such as gelatin, nidogen, collagen and heparan sulfate proteoglycans and mixtures thereof, K-carrageenan, poly(ethylene glycol) (PEG), polycaprolactone (PCL), poloxamer, peptide and mixtures thereof.

According to one embodiment, the crystallization trigger is a peptide, an oligopeptide or a protein, preferably the crystallization trigger is an oligopeptide, more preferably the crystallization trigger is an oligopeptide comprising 11 amino acid residues and comprising a hydrophobic aromatic core.

According to another embodiment, the crystallization trigger is an oligopeptide selected from the group comprising an oligopeptide of the HABP family, preferably HABP1 and HABP2, and an oligopeptide of the P11-family, preferably selected from the group consisting of P11-4, P11-8, P11-9, P11-12, P11-13, P11-14, P11-15, P11-16, P11-17, P11-18, P11-19, P11-20, P11-24, P11-25, P11-26, P11-27, P11-28-P11-29, P11-30, P11-31, P11-32 and mixtures, and most preferably P11-4, optionally the oligopeptide of the P11-family is associated with a negatively charged polysaccharide or a positively charged polysaccharide.

According to yet another embodiment, the crystallization trigger is an oligopeptide in which the amino acids at positions 4 and 8 are phenylalanine (F) and the amino acid at position 6 is tryptophan (W) and/or the amino acid residues at both positions 5 and 7 of the peptide are ornithine (O) or glutamic acid (E).

According to one embodiment, the ink comprises
a) the calcium cation-based compound in an amount ranging from 2 to 30 wt.-%, preferably from 5 to 25 wt.-%, and most preferably from 10 to 25 wt.-%, based on the total weight of the ink, and
b) the carrier material in an amount ranging from 1 to 10 wt.-%, preferably from 1 to 8 wt.-%, and most preferably from 1 to 5 wt.-%, based on the total weight of the ink, and
c) the crystallization trigger in an amount ranging from 0.05 to 3 wt.-%, preferably from 0.05 to 2.8 wt.-%, and most preferably from 0.1 to 2.5 wt.-%, based on the total weight of the ink, and
d) a buffer solution in an amount ranging from 57 to 96.95 wt.-%, preferably from 64.2 to 93.95 wt.-%, and most preferably from 67.5 to 98.9 wt.-%, based on the total weight of the ink.

According to one embodiment, the calcium cation-based compound being metastable calcium carbonate or metastable calcium phosphate comprises a stabilizer.

According to another embodiment, the stabilizer is selected from the group comprising magnesium chloride, polyaspartic acid, glutamic acid, polyacrylic acid, phosphates such as L-O-phosphoserine, sodium dihydrogen phosphate and disodium hydrogen phosphate, saccharides, EDTMP, xanthan, polysorbate, citric acid, ehylenediamine, extracts from biogenic samples, double-hydrophilic block copolymers and mixtures thereof.

According to a further aspect, a method for the production of a biomimetic minerizable 3D-printing ink, as defined herein, is provided, the method comprising the steps of
a) providing a calcium cation-based compound being metastable calcium carbonate or metastable calcium phosphate,
b) providing a carrier material,
c) providing a crystallization trigger, and
d) mixing the calcium cation-based compound being metastable calcium carbonate or metastable calcium phosphate of step a), the carrier material of step b) and the crystallization trigger of step c).

According to another aspect, a method for the production of a biomineralized 3D-printed article is provided, the method comprising the steps of
a) providing a biomimetic minerizable 3D-printing ink as defined herein,
b) printing the biomimetic minerizable 3D-printing ink into a predetermined form by using a 3D-printer, and
c) hardening the biomimetic minerizable 3D-printing ink at a temperature ranging from 10 to 50° C., preferably from 15 to 45° C., for obtaining the biomineralized 3D-printed article.

According to one embodiment, the hardening in step c) is carried out at
a) a temperature ranging from 15 to 40° C. and most preferably at a temperature ranging from 20 to 40° C., and/or
b) a hardening time ranging from 2 hours to 21 days, preferably from 12 hours to 19 days and most preferably from 24 hours to 18 days, and/or
c) a CO₂ content in the atmosphere from 3 to 6 vol.-%, preferably 4 to 6 vol-% and most preferably from 4.5 to 5.5 vol-%, and/or
d) a humidity of more than 75 vol. %, preferably in the range from 80 to 100 vol. % and most preferably in the range from 85 to 99.5 vol.-%.

According to another embodiment, the method comprises a further step d) of drying the biomineralized 3D-printed article obtained in step c), preferably at a temperature of at least 50° C., more preferably at a temperature ranging from 60 to 100° C. and most preferably from 65 to 90° C., and/or a drying time ranging from 5 hours to 36 hours, preferably from 5 hours to 32 hours and most preferably from 6 hours to 26 hours.

According to another aspect, a biomineralized 3D-printed article obtainable by a method as defined herein is provided.

According to one embodiment, the article is a dental reconstruction material, ceramic substitute, mollusk shell substitute, nacre substitute, dentin substitute, tooth substitute or bone substitute.

According to a further aspect, the use of a crystallization trigger which is an oligopeptide selected from the group comprising an oligopeptide of the HABP family and an oligopeptide of the P11-family for 3D printing is provided.

It should be understood that for the purpose of the present invention the following terms have the following meaning.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun, e.g. “a”, “an” or “the”, this includes a plural of that noun unless something else is specifically stated.

Terms like “obtainable” or “definable” and “obtained” or “defined” are used interchangeably. This e.g. means that, unless the context clearly dictates otherwise, the term “obtained” does not mean to indicate that e.g. an embodiment must be obtained by e.g. the sequence of steps following the term “obtained” though such a limited understanding is always included by the terms “obtained” or “defined” as a preferred embodiment.

According to the present invention, the biomimetic minerizable 3D-printing ink comprises
a) a calcium cation-based compound being metastable calcium carbonate or metastable calcium phosphate,
b) a carrier material, and
c) a crystallization trigger.

It has been especially found out that the biomimetic minerizable 3D-printing ink according to the present invention must comprise a calcium cation-based compound being metastable calcium carbonate or metastable calcium phosphate, a carrier material and a crystallization trigger.

In the following, it is referred to further details of the present invention and especially the foregoing biomimetic minerizable 3D-printing ink.

One requirement of the present invention is that the biomimetic minerizable 3D-printing ink comprises a calcium cation-based compound being metastable calcium carbonate or metastable calcium phosphate.

It is appreciated that the term “metastable” calcium carbonate refers to a calcium carbonate having a low content of or no crystalline morphology when analysed by known means of investigating a material's morphological state. For example, the metastable calcium carbonate has a crystallinity of less than 50 wt.-%, preferably of less than 40 wt.-%, more preferably of less than 30 wt.-% and most preferably of less than 20 wt.-%, based on the total weight of the calcium carbonate. Thus, the metastable calcium carbonate is preferably amorphous calcium carbonate.

Additionally, the “metastable” calcium carbonate thus has a high content of amorphous morphology when analysed by known means of investigating a material's morphological state. For example, the metastable calcium carbonate has an amorphous phase of more than 50 wt.-%, preferably of more than 60 wt.-%, more preferably of more than 70 wt.-% and most preferably of more than 80 wt.-%, based on the total weight of the calcium carbonate.

Alternatively, the term “metastable” calcium carbonate refers to a calcium carbonate having a high content of or consists of crystalline morphology when analysed by known means of investigating a material's morphological state, which is not stable and thus transforms into a more stable calcium carbonate species. For example, the metastable calcium carbonate has a crystallinity of more than 50 wt.-%, preferably of more than 70 wt.-%, more preferably of more than 80 wt.-% and most preferably of more than 90 wt.-%, e.g. about 100 wt.-%, based on the total weight of the calcium carbonate. Thus, the metastable calcium carbonate is preferably vaterite. For example, vaterite that is transformed into calcite.

Similarly, the term “metastable” calcium phosphate refers to a calcium phosphate having a low content of or no crystalline morphology when analysed by known means of investigating a material's morphological state. For example, the metastable calcium phosphate has a crystallinity of less than 50 wt.-%, preferably of less than 40 wt.-%, more preferably of less than 30 wt.-% and most preferably of less than 20 wt.-%, based on the total weight of the calcium phosphate. Thus, the metastable calcium phosphate is preferably amorphous calcium phosphate.

Additionally, the “metastable” calcium phosphate thus has a high content of amorphous morphology when analysed by known means of investigating a material's morphological state. For example, the metastable calcium phosphate has an amorphous phase of more than 50 wt.-%, preferably of more than 60 wt.-%, more preferably of more than 70 wt.-% and most preferably of more than 80 wt.-%, based on the total weight of the calcium phosphate.

Generally, the calcium cation-based compound thus has a crystallinity of less than 50 wt.-%, preferably of less than 40 wt.-%, more preferably of less than 30 wt.-% and most preferably of less than 20 wt.-%, based on the total weight of the calcium cation-based compound.

Additionally, the calcium cation-based compound thus has a high content of amorphous morphology when analysed by known means of investigating a material's morphological state. For example, the calcium cation-based compound has an amorphous phase of more than 50 wt.-%, preferably of more than 60 wt.-%, more preferably of more than 70 wt.-% and most preferably of more than 80 wt.-%, based on the total weight of the calcium cation-based compound.

In order to maintain the high content of amorphous phase, it is preferred that the calcium cation-based compound has low moisture content. According to one embodiment of the present invention, the calcium cation-based compound has a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the calcium cation-based compound.

For example, the calcium cation-based compound is metastable calcium carbonate having a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the metastable calcium carbonate. In one embodiment, the calcium cation-based compound is amorphous calcium carbonate having a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the metastable calcium carbonate. In another embodiment, the calcium cation-based compound is vaterite having a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the metastable calcium carbonate.

Alternatively, the calcium cation-based compound is metastable calcium phosphate having a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the metastable calcium phosphate. In one embodiment, the calcium cation-based compound is amorphous calcium phosphate having a moisture content of s 10.0 wt.-%, preferably from 0.01 to 10.0 wt.-%, more preferably from 0.02 to 9.0 wt.-% and most preferably from 0.05 to 8.0 wt.-%, based on the total dry weight of the metastable calcium phosphate.

It is preferred that the calcium cation-based compound has a particularly small particle size, preferably in the nanometer range. In particular, the particle size of the calcium cation-based compound is controlled as it is advantageous for the formation of the crystals and has an advantageous effect on the curing, which can proceed by generating an appropriate range of heat in the ink.

In one embodiment, the calcium cation-based compound thus preferably has a weight median particle size d₅₀ in the range from 1 to 500 nm. For example, the calcium cation-based compound has a weight median particle size d₅₀ in the range from 50 to 400 nm and most preferably from 100 to 350 nm.

In one embodiment, the calcium cation-based compound is metastable calcium carbonate having a weight median particle size d₅₀ in the range from 1 to 500 nm, preferably from 50 to 400 nm and most preferably from 100 to 350 nm.

In an alternative embodiment, the calcium cation-based compound is metastable calcium phosphate having a weight median particle size d₅₀ in the range from 1 to 500 nm, preferably from 50 to 400 nm and most preferably from 100 to 350 nm.

The value dₓ represents the diameter relative to which x % of the particles have diameters less than dₓ. This means that the d₅₀ value is the particle size at which 50% of all particles are smaller. The dₓ values are given in weight percent if not otherwise indicated. The d₅₀ value is thus the weight median particle size, i.e. 50 wt % of all grains are smaller than this particle size.

It is further to be noted that the weight median particle size d₅₀ in the meaning of the present invention refers to the primary particle size. That is to say, the calcium cation-based compound being metastable calcium carbonate or metastable calcium phosphate is deagglomerated, preferably by grinding. Methods for deagglomerating solid agglomerated materials such as the calcium cation-based compound are well known in the art and each method well known for this purpose can be used for the present invention. Preferably, such deagglomerating is carried out by using grinding methods optionally followed by a sieving in order to remove larger particles.

Additionally or alternatively, the calcium cation-based compound being metastable calcium carbonate or metastable calcium phosphate has a BET specific surface area in the range from 1.0 m²/g to 100.0 m²/g, more preferably from 5.0 m²/g to 80.0 m²/g and most preferably from 10.0 m²/g to 70.0 m²/g, measured using nitrogen and the BET method according to ISO 9277. For example, the calcium cation-based compound being metastable calcium carbonate has a BET specific surface area in the range from 10.0 m²/g to 70.0 m²/g, more preferably from 10.0 m²/g to 50.0 m²/g and most preferably from 15.0 m²/g to 40.0 m²/g, measured using nitrogen and the BET method according to ISO 9277. For example, the calcium cation-based compound being metastable calcium phosphate has a BET specific surface area in the range from 10.0 m²/g to 70.0 m²/g, more preferably from 25.0 m²/g to 70.0 m²/g and most preferably from 50.0 m²/g to 70.0 m²/g, measured using nitrogen and the BET method according to ISO 9277.

It is appreciated that the calcium cation-based compound being metastable calcium carbonate or metastable calcium phosphate can be obtained by any method known in the art. Preferably, the calcium cation-based compound being metastable calcium carbonate or metastable calcium phosphate is obtained by a method including the precipitation of the calcium cation-based compound being metastable calcium carbonate or metastable calcium phosphate in aqueous solution. For example, the precipitation can be carried out at a temperature ranging from 4 to 15° C., more preferably at a temperature ranging from 6 to 10° C. Additionally or alternatively, it is preferred that the precipitation is carried out at a pH equal to or above 7, more preferably at a pH ranging from 7 to 10. Additionally or alternatively, it is preferred that the concentrations of the compounds used for precipitation can vary from 0.02 mM to 2 M, preferably, from 0.2 mM to 1 M.

It is appreciated that the precipitation is carried out as fast as possible such that it is preferred that the compounds used for precipitation are contacted in one step with each other.

In one embodiment, the calcium cation-based compound being metastable calcium carbonate or metastable calcium phosphate is obtained by a method as described in the examples herein.

For example, if the calcium cation-based compound is metastable calcium phosphate, the metastable calcium phosphate can be obtained by precipitating of calcium phosphate salts in aqueous solution. The precipitation can be performed in that an aqueous solution of diammonium phosphate is contacted in one step with a calcium nitrate tetrahydrate aqueous solution. In a further step, the precipitated metastable calcium phosphate can be separated from the liquid phase, e.g. by filtration. The obtained metastable calcium phosphate may be subjected to a further step of cleaning, e.g. by freeze drying.

For example, if the calcium cation-based compound is metastable calcium carbonate, the metastable calcium carbonate can be obtained by precipitating of calcium carbonate salts in aqueous solution. The precipitation can be performed in that an aqueous solution of sodium carbonate is contacted in one step with a calcium chloride aqueous solution. In a further step, the precipitated metastable calcium carbonate can be separated from the liquid phase, e.g. by filtration. The obtained metastable calcium carbonate may be subjected to a further step of cleaning, e.g. by freeze drying.

It is further required that the biomimetic minerizable 3D-printing ink comprises a carrier material. It is specifically to be noted that the carrier material provides a matrix in which the calcium cation-based compound and the crystallization trigger are dispersed. Additional benefits of the carrier are the entanglement of the calcium cation-based compound and the crystallization trigger so that the solid content for the slurry does not separate from the liquid during extrusion. In addition thereto, the carrier gives structural support to the 3D-printed structure as well as offering the possibility to tune the viscosity of the ink.

In particular, any carrier material that is typically used in the kind of materials to be prepared can be used as carrier material in the present biomimetic minerizable 3D-printing ink. As the articles to be prepared are especially used as dental reconstruction material, ceramic substitute, mollusk shell substitute, nacre substitute, dentin substitute, tooth substitute or bone substitute, it is specifically preferred that the carrier material is biocompatible, has excellent hydrophilicity, no biotoxicity, and no immunogenicity. For example, carrier materials approved by the FDA for use as human body additives.

In one embodiment, the carrier material is a material suitable to form a hydrogel. The carrier material is thus preferably a material that is three-dimensionally crosslinked and does not disintegrate in water. Furthermore, the carrier material affects an increase of viscosity, but a decrease of flowability. In addition thereto, the carrier material is preferably pH buffered.

For example, the carrier material is selected from the group comprising gelatin, methylcellulose, alginate, agarose, fibrin, hyaluronic acid, proteins such as gelatin, nidogen, collagen and heparan sulfate proteoglycans and mixtures thereof, K-carrageenan, poly(ethylene glycol) (PEG), polycaprolactone (PCL), poloxamer, peptide and mixtures thereof.

Thus, it is appreciated that the carrier material is preferably a material suitable to form a hydrogel and is selected from the group comprising gelatin, methylcellulose, alginate, agarose, fibrin, hyaluronic acid, proteins such as gelatin, nidogen, collagen and heparan sulfate proteoglycans and mixtures thereof, K-carrageenan, poly(ethylene glycol) (PEG), polycaprolactone (PCL), poloxamer, peptide and mixtures thereof.

It is appreciated that the carrier material may be also a mixture of the above-mentioned materials. For example, the carrier material may be a mixture of proteins. For example, the carrier material may be a mixture of proteins such as gelatin, nidogen, collagen and heparan sulfate proteoglycans. Such a carrier material is well known in the art and is for example available under the tradename Matrigel™.

In view of the above, the carrier material may be a naturally derived hydrogel or synthetic based hydrogel.

More precisely, if the carrier material is a naturally derived hydrogel, the carrier material is preferably selected from the group comprising, more preferably consisting of, gelatin, methylcellulose, alginate, agarose, fibrin, hyaluronic acid, proteins such as gelatin, nidogen, collagen and heparan sulfate proteoglycans and mixtures thereof, K-carrageenan and mixtures thereof.

Alternatively, if the carrier material is a naturally derived hydrogel, the carrier material is preferably selected from the group comprising, more preferably consisting of, poly(ethylene glycol) (PEG), polycaprolactone (PCL), poloxamer, peptide and mixtures thereof.

As the articles to be prepared are especially used as dental reconstruction material, ceramic substitute, mollusk shell substitute, nacre substitute, dentin substitute, tooth substitute or bone substitute, it is preferred that the carrier material is a naturally derived hydrogel.

Most preferably, the carrier material is methylcellulose.

The biomimetic minerizable 3D-printing ink comprises a crystallization trigger. The crystallization trigger is specifically advantageous because it controls the crystallization of the calcium cation-based compound being metastable calcium carbonate or metastable calcium phosphate such that a controlled hardening of the biomimetic minerizable 3D-printing ink is obtained.

It is preferred that the crystallization trigger is a peptide, an oligopeptide or a protein. More preferably the crystallization trigger is an oligopeptide. For example, the crystallization trigger is an oligopeptide selected from the group comprising an oligopeptide of the HABP family, preferably HABP1 and HABP2.

Most preferably, the crystallization trigger is an oligopeptide comprising 11 amino acid residues and comprising a hydrophobic aromatic core. In one embodiment, the crystallization trigger is an oligopeptide selected from the group comprising, preferably consisting of, an oligopeptide of the HABP family and an oligopeptide of the P11-family. For example, the crystallization trigger is an oligopeptide of the P11-family.

In one embodiment of the invention, the oligopeptide comprising 11 amino acid residues and comprising a hydrophobic aromatic core has an overall net positive or negative charge and more preferably has an overall net even number charge of for example −6, −4, −2, +2, +4 or +6. Most preferably, the oligopeptide comprising 11 amino acid residues and comprising a hydrophobic aromatic core has an overall net negative charge and more preferably has an overall net even number charge of for example −6, −4, or −2.

For example, the oligopeptide is selected from the group consisting of P11-4 (sequence: CH3CO-QQRFEWEFEQQ-NH2), P11-8 (sequence: CH3CO-QQRFOWOFEQQ-NH2), P11-9 (sequence: CH3CO-SSRFEWEFESS-NH2), P11-12 (sequence: CH3CO-SSRFOWOFESS-NH2), P11-13 (sequence: CH3CO-EQEFEWEFEQE-NH2), P11-14 (sequence: CH3CO-QQOFOWFOQQ-NH2), P11-15 (sequence: CH3CO-NNRFEWEFENN-NH2), P11-16 (sequence: CH3CO-NNRFOWOFENN-NH2), P11-17 (sequence: CH3CO-TTRFEWEFETT-NH2), P11-18 (sequence: CH3CO-TTRFOWOFETT-NH2), P11-19 (sequence: CH3CO-QQRQOQOQEQQ-NH2), P11-20 (sequence: CH3CO-QQRQEQEQEQQ-NH2), P11-24 (sequence: CH30H-SSRQEQEQESS-NH2), P11-25 (sequence: CH3CO-SSRSESESESS-NH2), P11-26 (sequence: CH3CO-QQOQOQOQOQQ-NH2), P11-27 (sequence: CH3CO-EQEQEQEQEQE-NH2), P11-28 (sequence: CH3CO-OQOFOWOFOQO-NH2), P11-29 (sequence: CH3CO-QQEFEWEFEQQ-NH2), P11-30 (sequence: CH3CO-ESEFEWEFESE-NH2), P11-31 (sequence: CH3CO—SSOFOWOFOSS—NH2), P11-32 (sequence: CH3CO—OSOFOWOFOSO—NH2) and mixtures thereof.

In one embodiment, the oligopeptide of the P11-family is positively charged and has an overall net even number charge of for example +2, +4 or +6.

It is appreciated that the positively charged oligopeptides of the P11-family share sequence homology.

Preferably, the amino acid residues in positions 5 and 7 are ornithine (O). Preferably, the amino acid residues at positions 1 and 2 are the same but reversed at positions 10 and 11.

Preferably, the amino acid residues at positions 1 and 2 are the same and are selected from the group comprising serine (SS), glutamine (QQ), threonine (TT) and asparagine (NN).

Preferably, the amino acid residues at positions 10 and 11 are the same and are selected from the group comprising serine (SS), glutamine (QQ), threonine (TT) and asparagine (NN).

In some embodiments, the amino acid residues at positions 1 and 2 and 10 and 11 are the same so that they are all either serine (SS), glutamine (QQ), threonine (TT) or asparagine (NN).

Preferably, the amino acid residue at position 3 is either ornithine (O) or arginine (R).

Preferably, the amino acid residue at position 4 is either phenylalanine (F) or glutamine (Q).

Preferably, the amino acid residue at position 6 is either tryptophan (W) or glutamine (Q).

Preferably, the amino acid residue at position 8 is either phenylalanine (F) or glutamine (Q).

Preferably, the amino acid residue at position 9 is either glutamic acid (E) or glutamine (Q).

In one embodiment, the positively charged oligopeptide is selected from the group consisting of P11-8 (sequence: CH3CO-QQRFOWOFEQQ-NH2), P11-12 (sequence: CH3CO-SSRFOWOFESS-NH2), P11-14 (sequence: CH3CO-QQOFOWFOQQ-NH2), P11-15 (sequence: CH3CO-NNRFEWEFENN-NH2), P11-16 (sequence: CH3CO-NNRFOWOFENN-NH2), P11-18 (sequence: CH3CO-TTRFOWOFETT-NH2), P11-19 (sequence: CH3CO-QQRQOQOQEQQ-NH2), P11-26 (sequence: CH3CO-QQOQOQOQOQQ-NH2), P11-28 (sequence: CH3CO-OQOFOWOFOQO-NH2), P11-31 (sequence: CH3CO—SSOFOWOFOSS—NH2), P11-32 (sequence: CH3CO—OSOFOWOFOSO—NH2) and mixtures thereof.

Preferably, the positively charged oligopeptide is selected from the group consisting of P11-14 (sequence: CH3CO-QQOFOWFOQQ-NH2), P11-28 (sequence: CH3CO-OQOFOWOFOQO-NH2), P11-31 (sequence: CH3CO—SSOFOWOFOSS—NH2), and mixtures thereof.

It is appreciated that the amino acid chain of the oligopeptide may be extended to include a bioactive peptide sequence, or wherein the amino acid chain is attached to a therapeutically active molecule or drug or the like.

In one embodiment, the oligopeptide of the P11-family is positively charged and is associated with a negatively charged polysaccharide.

Preferably, the polysaccharide is selected from the group comprising glycosaminoglycan, oligosaccharide, mucopolysaccharide and dextran. Preferably, the oligopeptide of the P11-family is complexed with an oppositely charged polysaccharide (e.g. a negatively charged polysaccharide). In one embodiment, the negatively charged polysaccharide is a glycosaminoglycan (GAG) and is selected from the group comprising chondroitin sulphates (CS), dermatan sulphates, keratan sulphates, hyaluronan, hyaluronic acid, heparin and heparan sulphate or derivatives thereof. The polysaccharide may be natural or synthetic. The peptide complex may be selected from a SAP: polysaccharide combination, a SAP:oligosaccharide combination and a SAP:GAG combination. In particular, the peptide complex is a SAP:GAG combination.

For example, the oligopeptide of the P11-family is positively charged peptide and is associated with a polysaccharide selected from the group comprising glycosaminoglycan (GAG), oligosaccharide, mucopolysaccharide and dextran.

Throughout this application, the complex of the oligopeptide of the P11-family and charged polysaccharide may be referred to as e.g. a peptide:glycan or a peptide:GAG or P11:glycan or P11:GAG.

In one embodiment, the oligopeptide is selected from the group consisting of P11-8 (sequence: CH3CO-QQRFOWOFEQQ-NH2):GAG, P11-12 (sequence: CH3CO-SSRFOWOFESS-NH2):GAG, P11-14 (sequence: CH3CO-QQOFOWFOQQ-NH2):GAG, P11-15 (sequence: CH3CO-NNRFEWEFENN-NH2):GAG, P11-16 (sequence: CH3CO-NNRFOWOFENN-NH2):GAG, P11-18 (sequence: CH3CO-TTRFOWOFETT-NH2):GAG, P11-19 (sequence: CH3CO-QQRQOQOQEQQ-NH2):GAG, P11-26 (sequence: CH3CO-QQOQOQOQOQQ-NH2):GAG, P11-28 (sequence: CH3CO-OQOFOWOFOQO-NH2):GAG, P11-31 (sequence: CH3CO—SSOFOWOFOSS—NH2):GAG, P11-32 (sequence: CH3CO—OSOFOWOFOSO—NH2):GAG and mixtures thereof.

For example, the positively charged oligopeptide is selected from the group consisting of P11-14 (sequence: CH3CO-QQOFOWFOQQ-NH2):GAG, P11-28 (sequence: CH3CO-OQOFOWOFOQO-NH2):GAG, P11-31 (sequence: CH3CO—SSOFOWOFOSS—NH2):GAG, and mixtures thereof.

Alternatively, the oligopeptide of the P11-family is negatively charged and has an overall net even number charge of for example −2, −4 or −6. Preferably, the negatively charged oligopeptide of the P11-family has an overall net negative charge of −2.

It is appreciated that the negatively charged oligopeptides of the P11-family share sequence homology.

Preferably, the amino acid residues at positions 5, 7 and 9 are glutamic acid (E).

Preferably, the amino acid residue at position 3 is either glutamic acid (E) or arginine (R).

Preferably, the amino acid residue at positions 4 and 8 are the same and are preferably selected from the group consisting of phenylalanine (F), glutamine (Q) and serine (S). Preferably, the amino acid residue at position 6 is selected from the group consisting of tryptophan (W), glutamine (Q) and serine (S).

Preferably, the amino acid residues at positions 1 and 2 may be the same or different and reversed at positions 10 and 11.

Preferably, the amino acid residues at positions 1 and 2 are the same and are selected from the group comprising serine (SS), glutamine (QQ), threonine (TT) and asparagine (NN).

Preferably, the amino acid residues at positions 10 and 11 are the same and are selected from the group comprising serine (SS), glutamine (QQ), threonine (TT) and asparagines (NN).

In one embodiment, the crystallization trigger is an oligopeptide in which the amino acids at positions 4 and 8 are phenylalanine (F) and the amino acid at position 6 is tryptophan (W).

Additionally or alternatively, the crystallization trigger is an oligopeptide in which the amino acid residues at both positions 5 and 7 of the peptide are ornithine (O) or glutamic acid (E).

For example, the crystallization trigger is an oligopeptide in which the amino acids at positions 4 and 8 are phenylalanine (F) and the amino acid at position 6 is tryptophan (W) or the amino acid residues at both positions 5 and 7 of the peptide are ornithine (O) or glutamic acid (E).

Preferably, the crystallization trigger is an oligopeptide in which the amino acids at positions 4 and 8 are phenylalanine (F) and the amino acid at position 6 is tryptophan (W) and the amino acid residues at both positions 5 and 7 of the peptide are ornithine (O) or glutamic acid (E).

For example, the oligopeptide is selected from the group consisting of P11-4 (sequence: CH3CO-QQRFEWEFEQQ-NH2), P11-9 (sequence: CH3CO-SSRFEWEFESS-NH2), P11-13 (sequence: CH3CO-EQEFEWEFEQE-NH2), P11-15 (sequence: CH3CO-NNRFEWEFENN-NH2), P11-17 (sequence: CH3CO-TTRFEWEFETT-NH2), P11-20 (sequence: CH3CO-QQRQEQEQEQQ-NH2), P11-24 (sequence: CH30H-SSRQEQEQESS-NH2), P11-25 (sequence: CH3CO-SSRSESESESS-NH2), P11-27 (sequence: CH3CO-EQEQEQEQEQE-NH2), P11-29 (sequence: CH3CO-QQEFEWEFEQQ-NH2), P11-30 (sequence: CH3CO-ESEFEWEFESE-NH2), and mixtures thereof.

Preferably, the oligopeptide is selected from the group consisting of P11-4 (sequence: CH3CO-QQRFEWEFEQQ-NH2), P11-13 (sequence: CH3CO-EQEFEWEFEQE-NH2), P11-29 (sequence: CH3CO-QQEFEWEFEQQ-NH2), P11-30 (sequence: CH3CO-ESEFEWEFESE-NH2), and
mixtures thereof.

Most preferably, the oligopeptide of the P11-family is P11-4 (sequence: CH3CO-QQRFEWEFEQQ-NH2).

It is appreciated that where the negatively charged oligopeptide of the P11-family is used it may be desired to use a positively charged polysaccharide such as chitosan and its derivatives or other cationic polysaccharides. Alternatively, where the complex comprises the positively charged oligopeptide of the P11-family it may be desired to use a positively charged polysaccharide (i.e. of the same charge) such as chitosan and its derivatives or other cationic polysaccharides.

Preferably, the oligopeptide of the P11-family has an elastic modulus in the range of 1 to 400,000 Pa.

The ratio of oligopeptide of the P11-family to GAG in the complexes is selected according to the required mechanical properties and also the viscosity required.

It is appreciated that the biomimetic minerizable 3D-printing ink preferably has a viscosity in the range from 200 to 800 Pa-s, measured on a conventional Brookfield viscometer, e.g., EV-2+ type with a disk spindle of 3 and 100 rpm.

Preferably, the oligopeptide of the P11-family forms a gel or gel like substance (hydrogel) within seconds up to 24 hours. It is to be noted that the time of forming the gel or gel like substance (hydrogel) depends on the buffer system used and can be selected according to the desired need. If the oligopeptide of the P11-family is associated with a polysaccharide selected from the group comprising glycosaminoglycan (GAG), the oligopeptide preferably form a gel or gel like substance (hydrogel) before the oligopeptide and GAG are mixed.

Also provided is a charged oligopeptide of the P11-family, wherein the amino acid chain is extended to include a bioactive peptide sequence, or wherein the amino acid chain is attached to a therapeutically active molecule or drug or the like.

It is to be noted that the crystallization trigger may also act as carrier material. In this case, the crystallization trigger and the carrier material are the same. For example, if the crystallization trigger act as the carrier material, i.e. the crystallization trigger and the carrier material are the same, the crystallization trigger and the carrier material are preferably collagen.

It is appreciated that the biomimetic minerizable 3D-printing ink may further comprise a buffer solution, preferably a buffer solution that is typically used in the kind of materials to be prepared. A buffer solution is advantageously used in order to buffer the ink in a pH range around 7 in order to avoid a dissolving of the calcium cation-based compound. Furthermore, the buffer solution is used to maintain all the possible charges in solution stable, including peptides, minerals additives and cells that can be present.

For example, the buffer solution may be selected from the group comprising PBS buffer solution, Tris buffer solution, citrate buffer solution, borate buffer solution and the like.

In view of the above, the biomimetic minerizable 3D-printing ink comprises
a) the calcium cation-based compound in an amount ranging from 2 to 30 wt.-%, based on the total weight of the ink, and
b) the carrier material in an amount ranging from 1 to 10 wt.-%, based on the total weight of the ink, and
c) the crystallization trigger in an amount ranging from 0.05 to 3 wt.-%, based on the total weight of the ink, and
d) the buffer solution in an amount ranging from 57 to 96.95 wt.-% based on the total weight of the ink.

For example, the biomimetic minerizable 3D-printing ink comprises
a) the calcium cation-based compound in an amount ranging from 5 to 25 wt.-%, and most preferably from 10 to 25 wt.-%, based on the total weight of the ink,
b) the carrier material in an amount ranging from 1 to 8 wt.-%, and most preferably from 1 to 5 wt.-%, based on the total weight of the ink, and
c) the crystallization trigger in an amount ranging from 0.05 to 2.8 wt.-%, and most preferably from 0.1 to 2.5 wt.-%, based on the total weight of the ink, and
d) the buffer solution in an amount ranging from 64.2 to 93.95 wt.-%, and most preferably from 67.5 to 98.9 wt.-%, based on the total weight of the ink.

In order to stabilize the morphology of the calcium cation-based compound being metastable calcium carbonate or metastable calcium phosphate, it is preferred that the calcium cation-based compound comprises a stabilizer.

Preferably, the stabilizer is selected from the group comprising magnesium chloride, polyaspartic acid, glutamic acid, polyacrylic acid, phosphates such as L-O-phosphoserine, sodium dihydrogen phosphate and disodium hydrogen phosphate, saccharides, EDTMP, xanthan, polysorbate, citric acid, ehylenediamine, extracts from biogenic samples, double-hydrophilic block copolymers and mixtures thereof.

Thus, in one embodiment, the biomimetic minerizable 3D-printing ink comprises, preferably consists of,
a) the calcium cation-based compound in an amount ranging from 2 to 30 wt.-%, based on the total weight of the ink, including the stabilizer,
b) the carrier material in an amount ranging from 1 to 10 wt.-%, based on the total weight of the ink, and
c) the crystallization trigger in an amount ranging from 0.05 to 3 wt.-%, based on the total weight of the ink, and
d) the buffer solution in an amount ranging from 57 to 96.95 wt.-% based on the total weight of the ink.

For example, the biomimetic minerizable 3D-printing ink comprises, preferably consists of,
a) the calcium cation-based compound in an amount ranging from 5 to 25 wt.-%, and most preferably from 10 to 25 wt.-%, based on the total weight of the ink, including the stabilizer,
b) the carrier material in an amount ranging from 1 to 8 wt.-%, and most preferably from 1 to 5 wt.-%, based on the total weight of the ink, and
c) the crystallization trigger in an amount ranging from 0.05 to 2.8 wt.-%, and most preferably from 0.1 to 2.5 wt.-%, based on the total weight of the ink, and
d) the buffer solution in an amount ranging from 64.2 to 93.95 wt.-%, and most preferably from 67.5 to 98.9 wt.-%, based on the total weight of the ink.

The biomimetic minerizable 3D-printing ink as defined herein is preferably prepared by a method for its production. The method for the production of the biomimetic minerizable 3D-printing ink comprises the steps of
a) providing a calcium cation-based compound being metastable calcium carbonate or metastable calcium phosphate,
b) providing a carrier material,
c) providing a crystallization trigger, and
d) mixing the calcium cation-based compound being metastable calcium carbonate or metastable calcium phosphate of step a), the carrier material of step b) and the crystallization trigger of step c).

With regard to the definition of the biomimetic minerizable 3D-printing ink, the calcium cation-based compound, the carrier material, the crystallization trigger and preferred embodiments thereof, reference is made to the statements provided above when discussing the technical details of the biomimetic minerizable 3D-printing ink of the present invention.

Step d) is performed by mixing the calcium cation-based compound being metastable calcium carbonate or metastable calcium phosphate of step a), the carrier material of step b) and the crystallization trigger of step c). It is appreciated that any mixing (or stirring) means may be used that is suitable to thoroughly mix the components with each other. Suitable equipment for mixing, agitation or stirring is known to the skilled person.

Step d) may be performed at room temperature, i.e. at a temperature of 20° C.±2° C., or at a temperature above the freezing point of the mixture prepared in step d). For example, step d) is performed at a temperature of 10 to 50° C., preferably 15 to 45° C.

If the calcium cation-based compound being metastable calcium carbonate or metastable calcium phosphate further comprises a stabilizer, the method for the production of the biomimetic minerizable 3D-printing ink comprises the steps of
a) providing a calcium cation-based compound being metastable calcium carbonate or metastable calcium phosphate comprising a stabilizer,
b) providing a carrier material,
c) providing a crystallization trigger, and
d) mixing the calcium cation-based compound being metastable calcium carbonate or metastable calcium phosphate comprising the stabilizer of step a), the carrier material of step b) and the crystallization trigger of step c).

### Biomineralized 3D-Printed Article and Method for the Production

According to a further aspect of the present invention, the biomimetic minerizable 3D-printing ink as defined herein can be used to prepare a biomineralized 3D-printed article.

Thus, the present invention further relates to a method for the production of a biomineralized 3D-printed article, the method comprising the steps of
a) providing a biomimetic minerizable 3D-printing ink,
b) printing the biomimetic minerizable 3D-printing ink into a predetermined form by using a 3D-printer, and
c) hardening the biomimetic minerizable 3D-printing ink at a temperature ranging from 10 to 50° C. for obtaining the biomineralized 3D-printed article.

With regard to the definition of the biomimetic minerizable 3D-printing ink, and preferred embodiments thereof, reference is made to the statements provided above when discussing the technical details of the biomimetic minerizable 3D-printing ink of the present invention.

As regards method step b), it is to be noted that any 3D-printer suitable for printing the articles to be prepared may be used for the present invention. For example, a 3D-printer may be selected from a pneumatic, extrusion and piston 3D printer. Such 3D-printer are well known to the skilled and do not need to be further described in detail in here.

In step c) of the method for the production of the biomineralized 3D-printed article, the biomimetic minerizable 3D-printing ink is hardened at a temperature ranging from 10 to 50° C. for obtaining the biomineralized 3D-printed article. For example, the biomimetic minerizable 3D-printing ink is hardened at a temperature ranging from 15 to 45° C. in step c) for obtaining the biomineralized 3D-printed article.

In one embodiment, the biomimetic minerizable 3D-printing ink is hardened at a temperature ranging from 15 to 40° C. and most preferably at a temperature ranging from 20 to 40° C., in step c).

Additionally or alternatively, the hardening in step c) is carried out at a hardening time ranging from 2 hours to 21 days, preferably from 12 hours to 19 days and most preferably from 24 hours to 18 days. For example, the hardening in step c) is carried out at a hardening time ranging from 2 hours to 5 days, preferably from 12 hours to 4 days and most preferably from 24 hours to 72 hours.

Additionally or alternatively, the hardening in step c) is carried out at a CO₂ content in the atmosphere from 3 to 6 vol.-%, preferably 4 to 6 vol-% and most preferably from 4.5 to 5.5 vol-%.

Additionally or alternatively, the hardening in step c) is carried out at a humidity of more than 75 vol. %, preferably in the range from 80 to 100 vol. % and most preferably in the range from 85 to 99.5 vol.-%.

In one embodiment, the hardening in step c) is thus carried out at
a) a temperature ranging from 15 to 40° C. and most preferably at a temperature ranging from 20 to 40° C., or
b) a hardening time ranging from 2 hours to 21 days, preferably from 12 hours to 19 days and most preferably from 24 hours to 18 days, or
c) a CO₂ content in the atmosphere from 3 to 6 vol.-%, preferably 4 to 6 vol-% and most preferably from 4.5 to 5.5 vol-%, or
d) a humidity of more than 75 vol. %, preferably in the range from 80 to 100 vol. % and most preferably in the range from 85 to 99.5 vol.-%.

Preferably, the hardening in step c) is carried out at high humidity resulting in the crystallization of the calcium cation-based compound being metastable calcium carbonate or metastable calcium phosphate. Thus, it is preferred that the hardening in step c) is carried out at
a) a temperature ranging from 10 to 50° C., preferably from 15 to 45° C., more preferably from 15 to 40° C. and most preferably at a temperature ranging from 20 to 40° C., and
b) a humidity of more than 75 vol. %, preferably in the range from 80 to 100 vol. % and most preferably in the range from 85 to 99.5 vol.-%.

It is further preferred that the CO₂ content in the atmosphere is kept in a specific range which advantageously influences the formation of cells and the mineralization of the samples. Thus, it is preferred that the hardening in step c) is carried out at
a) a temperature ranging from 10 to 50° C., preferably from 15 to 45° C., more preferably from 15 to 40° C. and most preferably at a temperature ranging from 20 to 40° C., and
b) a humidity of more than 75 vol. %, preferably in the range from 80 to 100 vol. % and most preferably in the range from 85 to 99.5 vol.-%, and
c) a CO₂ content in the atmosphere from 3 to 6 vol.-%, preferably 4 to 6 vol-% and most preferably from 4.5 to 5.5 vol-%.

It is appreciated that the hardening is preferably carried out for a time ranging from 2 hours to 21 days, preferably from 12 hours to 19 days and most preferably from 24 hours to 18 days. For example, the hardening is carried out for a time ranging from 2 hours to 5 days, preferably from 12 hours to 4 days and most preferably from 24 hours to 72 hours. Thus, it is preferred that the hardening in step c) is carried out at
a) a temperature ranging from 10 to 50° C., preferably from 15 to 45° C., more preferably from 15 to 40° C. and most preferably at a temperature ranging from 20 to 40° C., and
b) a humidity of more than 75 vol. %, preferably in the range from 80 to 100 vol. % and most preferably in the range from 85 to 99.5 vol.-%, and
c) a CO₂ content in the atmosphere from 3 to 6 vol.-%, preferably 4 to 6 vol-% and most preferably from 4.5 to 5.5 vol-%, and
d) a hardening time ranging from 2 hours to 21 days, preferably from 12 hours to 19 days and most preferably from 24 hours to 18 days, e.g. from 2 hours to 5 days, or from 12 hours to 4 days or from 24 hours to 72 hours.

In order to subject the obtained biomineralized 3D-printed article to a final hardening, the method preferably further comprises a step of drying the article at e.g. elevated temperature or for a sufficient time.

Thus, the method for the production of a biomineralized 3D-printed article preferably comprises a further step d) of drying the biomineralized 3D-printed article obtained in step c). In a preferred embodiment, the drying is carried out at a temperature of at least 50° C., more preferably at a temperature ranging from 60 to 100° C. and most preferably from 65 to 90° C., or for a drying time ranging from 5 hours to 36 hours, preferably from 5 hours to 32 hours and most preferably from 6 hours to 26 hours. For example, a drying time ranging from 5 hours to 24 hours, preferably from 5 hours to 18 hours and most preferably from 6 hours to 12 hours. Preferably, the method for the production of a biomineralized 3D-printed article preferably comprises a further step d) of drying the biomineralized 3D-printed article obtained in step c), preferably at a temperature of at least 50° C., more preferably at a temperature ranging from 60 to 100° C. and most preferably from 65 to 90° C.

It is appreciated that the final hardening preferably further comprises a step of drying the article at elevated temperature, which is, depending on the applied temperature, carried out for a sufficient time.

In one embodiment, the drying is carried out such that the article has a low residual moisture content. However, in view of the articles to be prepared it is advantageous to avoid a complete drying out of the article when drying in order to mimic nature and to keep cells alive. Thus, it is preferred that the drying results in an article having a residual moisture content as measured by TGA in the range from 1 to 8 wt.-%, preferably from 2 to 5 wt.-%.

Thus, the method comprises a further step d) of drying the biomineralized 3D-printed article obtained in step c), preferably at a temperature of at least 50° C., more preferably at a temperature ranging from 60 to 100° C. and most preferably from 65 to 90° C., and a drying time ranging from 5 hours to 36 hours, preferably from 5 hours to 32 hours and most preferably from 6 hours to 26 hours. For example, a drying time ranging from 5 hours to 24 hours, preferably from 5 hours to 18 hours and most preferably from 6 hours to 12 hours.

In view of this, the method for the production of the biomineralized 3D-printed article preferably comprises the steps of
a) providing a biomimetic minerizable 3D-printing ink,
b) printing the biomimetic minerizable 3D-printing ink into a predetermined form by using a 3D-printer,
c) hardening the biomimetic minerizable 3D-printing ink at a temperature ranging from 10 to 50° C. for obtaining the biomineralized 3D-printed article, and
d) drying the biomineralized 3D-printed article obtained in step c), preferably at a temperature of at least 50° C., more preferably at a temperature ranging from 60 to 100° C. and most preferably from 65 to 90° C., and a drying time ranging from 5 hours to 36 hours, preferably from 5 hours to 32 hours and most preferably from 6 hours to 26 hours.

According to a further aspect, the present invention also relates to a biomineralized 3D-printed article. The article is preferably obtainable by the method for the production of a biomineralized 3D-printed article as defined herein.

Thus, the biomineralized 3D-printed article is obtainable by a method for the production of a biomineralized 3D-printed article comprising the steps of
a) providing a biomimetic minerizable 3D-printing ink,
b) printing the biomimetic minerizable 3D-printing ink into a predetermined form by using a 3D-printer, and
c) hardening the biomimetic minerizable 3D-printing ink at a temperature ranging from 10 to 50° C. for obtaining the biomineralized 3D-printed article.

Preferably, the biomineralized 3D-printed article is obtainable by a method for the production of a biomineralized 3D-printed article comprising the steps of
a) providing a biomimetic minerizable 3D-printing ink,
b) printing the biomimetic minerizable 3D-printing ink into a predetermined form by using a 3D-printer, and
c) hardening the biomimetic minerizable 3D-printing ink at a temperature ranging from 10 to 50° C. for obtaining the biomineralized 3D-printed article, and
d) drying the biomineralized 3D-printed article obtained in step c), preferably at a temperature of at least 50° C., more preferably at a temperature ranging from 60 to 100° C. and most preferably from 65 to 90° C., and a drying time ranging from 5 hours to 36 hours, preferably from 5 hours to 32 hours and most preferably from 6 hours to 26 hours.

It is appreciated that the article is preferably a dental reconstruction material, ceramic substitute, mollusk shell substitute, nacre substitute, dentin substitute, tooth substitute or bone substitute.

In view of the advantageous effect obtained for the present invention, another aspect of the present application further refers to the use of a biomimetic minerizable 3D-printing ink for 3D printing.

With regard to the definition of the biomimetic minerizable 3D-printing ink, and preferred embodiments thereof, reference is made to the statements provided above when discussing the technical details of the biomimetic minerizable 3D-printing ink of the present invention.

The inventors found out that the crystallization trigger is of advantage to increase the hardness of the constructs once being 3D printed. Thus, a further aspect of the present invention relates to the use of a crystallization trigger which is an oligopeptide selected from the group comprising an oligopeptide of the HABP family and an oligopeptide of the P11-family for 3D printing.

Alternatively or additionally, the use of a calcium cation-based compound being metastable calcium carbonate or metastable calcium phosphate for 3D printing is provided.

The following examples and tests will illustrate the present invention, but are not intended to limit the invention in any way.

### BRIEF DESCRIPTION OF THE FIGURES

FIG. 1 shows SEM pictures of the prepared ACP (left) and ACP after printing (right)

FIG. 2 shows a SEM picture of the prepared ACC

FIG. 3 shows a hardness comparison for the prepared ACP inks

FIG. 4 shows the Vickers hardness for example 2

FIG. 5 shows the Vickers hardness for example 3

FIG. 6 shows the Vickers hardness for example 4

FIG. 7 shows a hardness comparison for the prepared ACC inks

FIG. 8 shows a XRD comparison between ACP before and after 3D printing example 4

FIG. 9 shows a FTIR comparison between ACP before and after 3D printeding example 4

### EXAMPLES

### Materials Used

CaCl₂*2H₂O≥99% of Sigma Aldrich
Na₂CO₃≥99.5% of Sigma Aldrich
L-O-phosphoserine ≥98% of TCI
Ca(NO₃)₂*4H₂O≥99% of Sigma Aldrich
(NH₄)₂HPO₄≥98% of Sigma Aldrich
NH₄OH-solution (28.0-30.0% NH₃ basis) of Sigma Aldrich
Whatman Cellulose Nitrate Membrane Filters, Ø9 cm, 0.8 μm
Fann filter, special hardened filter paper, Ø9 cm, 2-5 μm
Amorphous calcium phosphate ACP and amorphous calcium carbonate ACC: Omya AG
Methyl cellulose (MC) of Sigma Aldrich
NaOH of Sigma Aldrich
Na₂HPO₄ of Sigma Aldrich
Citric Acid of Sigma Aldrich
1×PBS (pH=7.4) of Sigma Aldrich
Peptide P11-4 (sequence: CH3CO-QQRFEWEFEQQ-NH2) of Credentis AG

### Preparation of ACP and ACC

ACP preparation: Precipitation of calcium phosphate salts in aqueous solution was performed in that an aqueous solution of diammonium phosphate was stirred, while adding in one step the calcium nitrate tetrahydrate aqueous solution. In this step, a precipitation was observed. The ACP was recovered by filtration and cleaning steps followed by freeze drying.

The details of the solution preparation and of the synthesis are set out in the following tables 1 and 2.

TABLE 1
solution preparation for ACP

| | Phosphate | Calcium | Rinse |
|---|---|---|---|
| | Solution | solution | Solution |
| | 0.15M | 0.35M | 0.14 wt % |
| (NH₄)₂HPO₄ [g] | 15.4 | — | — |
| Ca(NO₃)₂ *4H₂O [g] | — | 27.5 | — |
| NH₄OH (28-30 wt %) [ml] | 23.6 | 24.8 | 2.43 |
| Dest. water [ml] | 746.4 | 308.2 | 1797.57 |
| Total volume [ml] | 777 | 333 | 1800 |

TABLE 2
Synthesis of ACP

| | Phosphate | Calcium | |
|---|---|---|---|
| | solution | solution | |
| | 0.15M | 0.35M | |
| Mixing | 65 ml | 27.5 ml | Calcium solution was added to |

| | | |
|---|---|---|
| 1200 rpm, | | phosphate solution with stirring, |
| 30 sec | | precipitation took place. |
| Filtration | fann filter, Part Nr. 206051 | Precipitation was separated |
| | Ø 9 cm, pore size 2-5 μm | from liquid phase by |
| | approx. 15 min | filtration. |
| Rinsing | Rinse solution | filter cake was rinsed during |
| | 150 ml | filtration |
| Freeze | ~48 h | 1 mbar, −54° C. |
| drying | | Manually ground and sieved |
| Grinding | | in a 100 μm sieve |
| and | | |
| sieving | | |
| d₅₀ | | ~236 nm |
| BET SSA | | 64.69 m²/g |

The final products were stored in a desiccator until further uses.

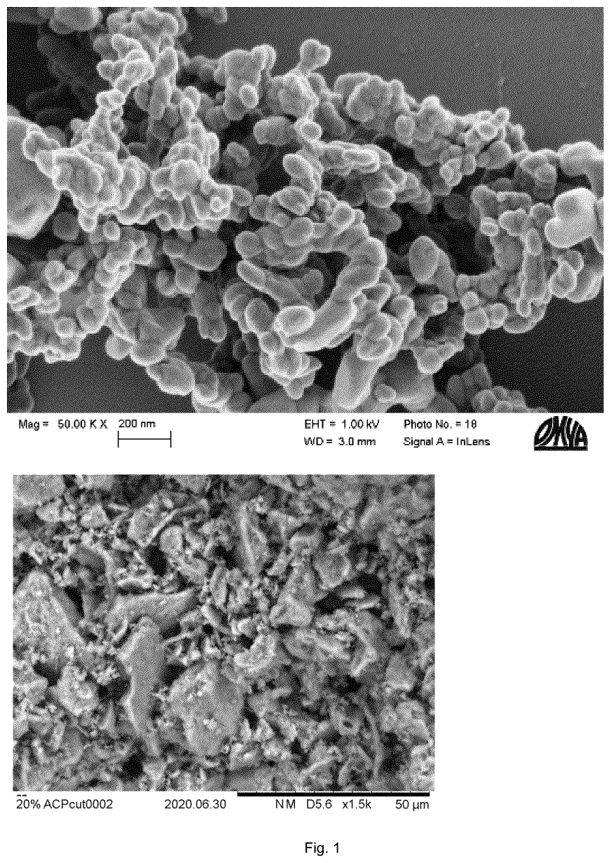

FIG. 1 shows SEM pictures of the prepared ACP (left) and ACP after printing (right).

ACC preparation: Precipitation of calcium carbonate salts in aqueous solution was performed in that an aqueous solution of sodium carbonate was stirred while adding in one step the calcium chloride aqueous solution. In this step, a precipitation was observed. The ACC was recovered by filtration and cleaning steps followed by freeze drying.

The details of the solution preparation and of the synthesis are set out in the following tables 3 and 4.

TABLE 3
solution preparation for ACC

| | Carbonate solution 0.25M | Calcium solution |
|---|---|---|
| | with stabilizer 0.08M | 0.25M |
| NaCO₃ [g] | 13.248 | — |
| CaCl₂ [g] | — | 18.376 |
| L-O-Phosphoserine [g] | 7.403 | — |
| MilliQ [ml] | 500 | 500 |
| Total Volume | 500 | 500 |
| pH | 9.38 | 6.95 |

TABLE 4
Synthesis of ACC

| | Carbonate | Calcium | |
|---|---|---|---|
| | Solution 0.25M | solution | |
| | (stabilizer) | 0.25M | |
| Mixing | 40 ml | 40 ml | Calcium solution (5° C.) was |

| | | |
|---|---|---|
| 1200 rpm, | | added to carbonate solution |
| 30 sec | | (5° C.) with stirring, |
| | | precipitation took place. |
| Filtration | Cellulose Nitrate Membrane | Precipitation was separated |
| | Ø 9 cm, pore size 0.8 μm | from liquid phase by |
| | approx. 15 min | filtration. |
| Rinsing | Cold miliQ (5° C.) | filter cake was rinsed during |
| | 50 ml | filtration |
| Freeze | ~48 h | 1 mbar, −54° C. |
| drying | | Manually ground and sieved |
| Grinding | | in a 100 μm sieve |
| and | | |
| sieving | | |
| d₅₀ | | ~332 nm |
| BET SSA | | 28.03 m²/g |

The final product was stored in a desiccator until further uses.

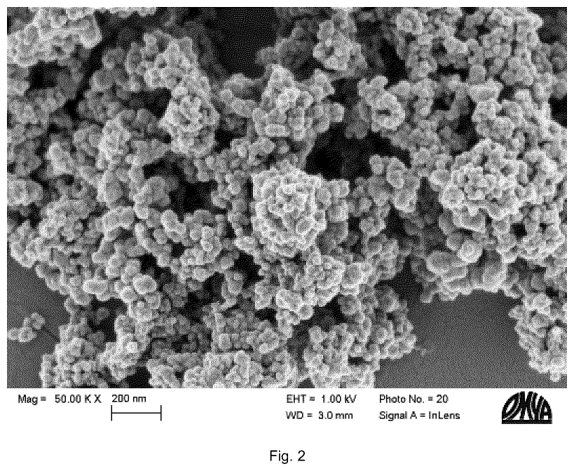

FIG. 2 shows a SEM picture of the prepared ACC.

### Preparation of Solutions

Stock Solution MC 15 cp (Methyl Cellulose) light (7.4% w/w)
Dissolve 8 g MC into 100 g PBS (Phosphate Buffered Saline) and let swell overnight at 4° C.
P11-4 Peptide (1%, 3% w/w) (for 3 ml)—adapted accordingly for different ml
Dissolve peptide P11-4 in a solution of 0.04 M Na₂HPO₄, 1 M NaOH and 0.02 M citric acid.

### Characterization Methods

### TGA

Thermal gravimetrical analysis was performed on a TGA 4000 from Perkin Elmer. The TGA program was hold for 1.0 min at 30.00° C. and heat from 30.00° C. to 900.00° C. at 10.00° C./min.

### BET

Powder specific surface area was measured with Micromeritics ASAP 2460. The degassing of the sample was done under vacuum. Samples were measured according to norm ISO 9277.

### Particle Size Distribution

Particle size distribution was performed by dynamic light scattering by using a Malvern Zetasizer Nano ZS. For the sample preparation, a dispersed solution containing 0.1 wt % of the particles in an aqueous solution and 0.0021 wt % of polyacrylate dispersant was prepared. The suspension was high shear mixed for 3 minutes and then ultrasonicated for 30 minute. The sample was measured.

### Vickers Experiment

The measurement was performed with Struers DURAMIN-40 M1 (https://www.struers.com/en/Products/Hardness-testing/Hardness-testing-equipment/Duramin-40).

To obtain a flat surface for indentation, the samples were grinded (1000 grade paper), polished (cotton cloth) and cleaned with a fiberless paper towel by hand. Surface was flushed with N₂ after the analysis. The indentation position was chosen by optical inspection and navigation with micrometer stage. Then, the load appropriate for HV0.01 was applied. The tip dwelled in the force load position for 10s before being retracted. The size of the indent was measured optically, followed by an automatic evaluation of the Vickers hardness.

The instruments works according to the following norms: ASTM E384, ISO 6507 and JIS Z 2244.

### XRD

About 0.16 cm³ of sample was loaded into a PMMA specimen holder, offering a flat, circular (d=20 mm) surface for XRD analysis. The sample was then analyzed obeying Bragg's law, using a Bruker D8 Advance ECO powder diffractometer with a 1 kW X-ray tube operating 40 kV and 25 mA, a Θ-Θ (Bragg-Brentano) goniometer, and a LYNXEYE XE-T detector, scanning from 3° to 70° 2Θ at 0.02° 2Θ steps for 0.5 s per step. Nickel-filtered Cu K_{α} radiation (λ=1.54060-10⁻¹⁰ m) was employed in all experiments. During measurement, the sample was rotated at 15 rpm to maximize random distribution of analyzed crystal surfaces. The resulting powder diffraction pattern was interpreted qualitatively with respect to mineral content using the Bruker DIFFRACsuite software package EVA in comparison to the ICDD PDF library of reference patterns.

### FTIR (Perkin-Elmer)

FTIR-spectra of the sample in attenuated total reflection (ATR) for type verification. The FTIR spectra were collected using a single bounce ATR unit (Gladi©-ATR). The spectral data were slightly smoothened (value 20), baseline corrected and normalized to 1.5 A (absorbance which corresponds to 3.16% transmission)

### Field Emission Scanning Electron Microscope (FESEM)

Field emission scanning electron microscope (Zeiss Sigma VP) was used for investigations. Two different detectors were involved:
1. Images from secondary electron detector (SE) show particle shape of the sample
2. InLens secondary electron detector (InLens) was used to show fine structures.

### EXAMPLES

Before the ink preparation, the gel liquid materials MC and peptides were prepared as follow:
1. 8 g of MC was dissolved in 100 g PBS (Phosphate Buffered Saline) and left to swell overnight at 4° C.
2. 90.63 mg of peptide P11-4 was dissolved in 100.6 μl of 1M NaOH/1.365 ml of 0.04 Na₂HPO₄. As final step for self-assembling, 1.5 ml of 0.02 M of citric acid was added.

For the ACP ink preparation, all the materials were mixed: 1^{st} the gel liquid materials (MC and P11-4) were mixed, followed by the addition of the mineral (ACP).

3D-printed article samples were prepared by using the 3D printer RegenHU 3Discovery via direct extrusion trough a metallic syringe needle (ID 0.3 mm) or conical PLA needle (ID 0.4 mm). The ink was filled in a 3 ml plastic syringe, tip was mounted. The ink was extruded by applying a pneumatic pressure until a flow rate of 1-2 mm/s was measured. Then, the 3D-printed article samples were printed in the form of squares of 1×1 cm line by line and layer by layer (each layer 3 mm high in total 3 layers) through computer controlled motion of the motorized 3D stages.

The 3D-printed article samples were stored in an incubator at 37° C., 99 vol-% humidity and 5 vol-% CO₂ for defined intervals of time (0.04, 017, 1, 2, 6, 9, 16 days) for hardening.

The 3D-printed article samples were further dried for 24 hours at 70° C. and stored in a desiccator for further characterization in Vickers, XRD and FTIR.

The 3D-printed article samples prepared are set out in the following table 5.

TABLE 5
3D-printed article samples prepared based on ACP

| | Example 1 | Example 2 | Example 3 | Example 4 |
|---|---|---|---|---|
| ACP (% w/w) | 20 | 20 | 20 | 20 |
| MC (% w/w) | 5.9 | 4.5 | 1.5 | 1.5 |
| P11-4 (% w/w) | — | 0.2 | 0.6 | 1.8 |
| Buffer aqueous | 74.1 | 75.3 | 77.9 | 76.7 |
| solution (% w/w) | | | | |
| Printability | +++ | ++ | +++ | +++ |

For the ACC ink preparation all the materials were mixed: 1^{st} the gel liquid materials (MC and P11-4) were mixed, followed by the addition of the mineral (ACC).

3D-printed article samples were prepared by using the 3D printer RegenHU 3Discovery via direct extrusion trough a metallic syringe needle (ID 0.3 mm) or conical PLA needle (ID 0.4 mm). The ink was filled in a 3 ml plastic syringe, tip was mounted. The ink was extruded by applying a pneumatic pressure until a flow rate of 1-2 mm/s was measured. Then, the 3D-printed article samples were printed in the form of squares of 1×1 cm line by line and layer by layer (each layer 3 mm high in total 3 layers) through computer controlled motion of the motorized 3D stages.

The 3D-printed article samples were stored in an incubator at 37° C., 99 vol-% humidity and 5 vol-% CO₂ for 16 days for hardening.

The 3D-printed article samples were further dried for 24 hours at 70° C. and stored in a desiccator for further characterization in Vickers, XRD and FTIR.

The 3D-printed article samples prepared are set out in the following table 6.

TABLE 6
3D-printed article samples prepared based on ACC

| | Example 5 | Example 6 |
|---|---|---|
| ACC (% w/w) | 20 | 20 |
| MC (% w/w) | 5.9 | 4.5 |
| P11-4 (% w/w) | — | 0.2 |
| Buffer aqueous | 74.1 | 75.3 |
| solution (% w/w) | | |
| Printability | +++ | ++ |

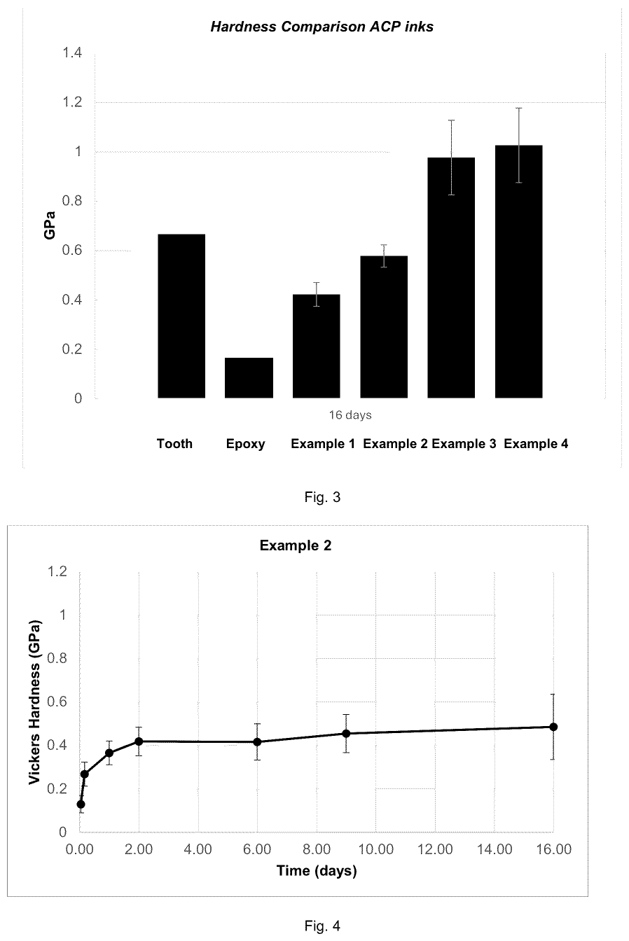

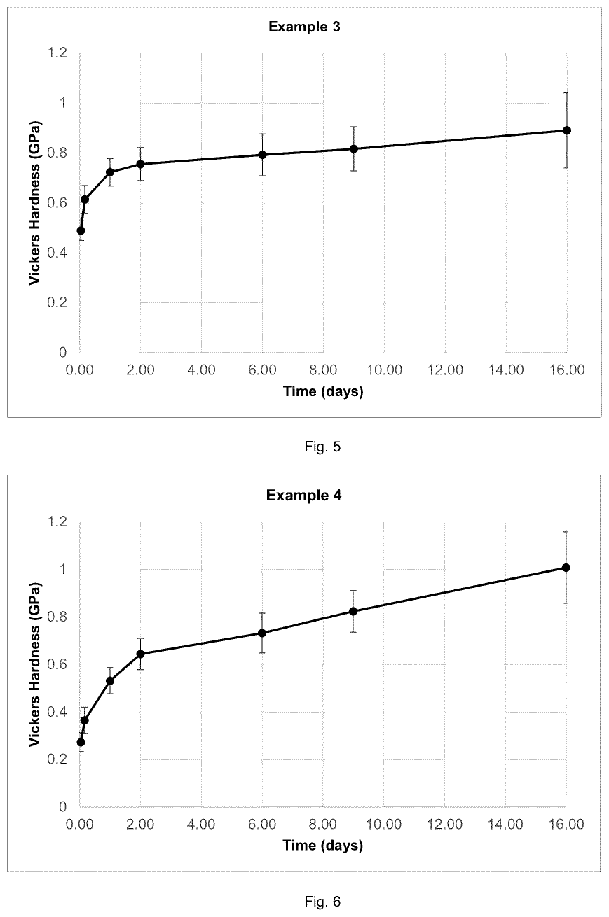

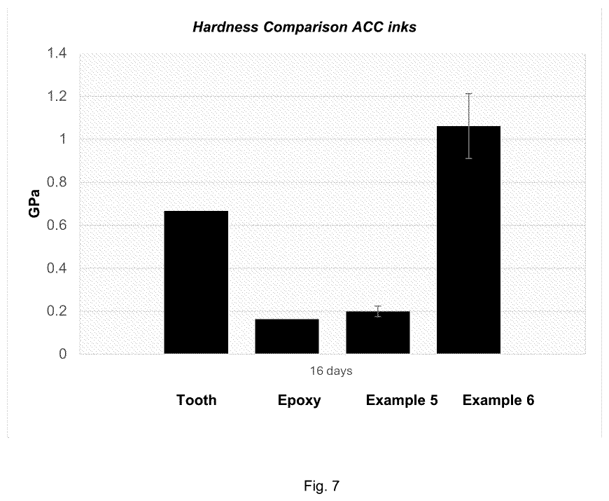

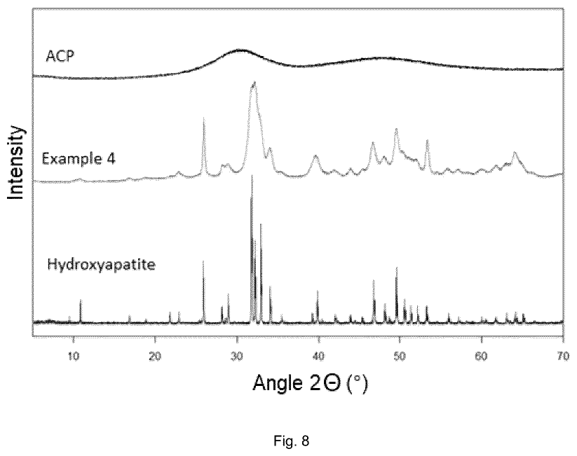

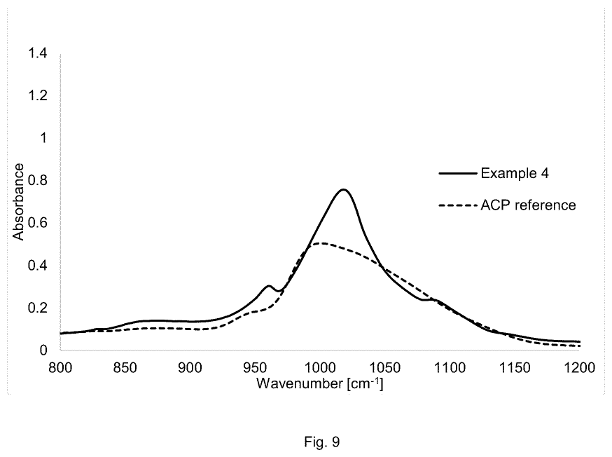

The use of a crystallization trigger such as a peptide is of advantage to increase the hardness of the constructs once being 3D printed. This was evidenced by the hardness measurements and comparison of the formulations that contain higher concentration of the crystallization trigger as can be seen in FIGS. 3, 4, 5, and 6, (examples 2, 3, 4 and 6). These samples show even higher hardness than the control (teeth). The time also plays a role on the increase of the hardness, this is possible to see on the higher crystallization trigger content formulations (example 3 and 4). This suggested again that the crystallization trigger is strongly involved in the hardening of the construct. It was also elucidated that the amorphous phase transformed towards the crystal phases, shown by XRD and FTIR (FIG. 8 and FIG. 9). In particular, the peak broadening shown for the ACP in FIG. 8 confirms that metastable calcium phosphate, and especially amorphous calcium phosphate, is present having a crystallinity of less than 20 wt.-%, based on the total weight of the calcium phosphate.

1. A biomimetic minerizable 3D-printing ink comprising
a) a calcium cation-based compound being metastable calcium carbonate or metastable calcium phosphate,
b) a carrier material, and
c) a crystallization trigger.

2. The biomimetic minerizable 3D-printing ink according to claim 1, wherein the calcium cation-based compound has a crystallinity of less than 50 wt.-%, preferably of less than 40 wt.-%, more preferably of less than 30 wt.-% and most preferably of less than 20 wt.-%, based on the total weight of the calcium cation-based compound.

3. The biomimetic minerizable 3D-printing ink according to claim 1, wherein the calcium cation-based compound has a weight median particle size d₅₀ as determined by dynamic light scattering in the range from 1 to 500 nm, preferably from 50 to 400 nm and most preferably from 100 to 350 nm.

4. The biomimetic minerizable 3D-printing ink according to claim 1, wherein the carrier material is a material suitable to form a hydrogel, preferably the carrier material is selected from the group comprising gelatin, methylcellulose, alginate, agarose, fibrin, hyaluronic acid, proteins such as gelatin, nidogen, collagen and heparan sulfate proteoglycans and mixtures thereof, K-carrageenan, poly(ethylene glycol) (PEG), polycaprolactone (PCL), poloxamer, peptide and mixtures thereof.

5. The biomimetic minerizable 3D-printing ink according to claim 1, wherein the crystallization trigger is a peptide, an oligopeptide or a protein, preferably the crystallization trigger is an oligopeptide, more preferably the crystallization trigger is an oligopeptide comprising 11 amino acid residues and comprising a hydrophobic aromatic core.

6. The biomimetic minerizable 3D-printing ink according to claim 1, wherein the crystallization trigger is an oligopeptide selected from the group comprising an oligopeptide of the HABP family, preferably HABP1 and HABP2, and an oligopeptide of the P11-family, preferably selected from the group consisting of P11-4, P11-8, P11-9, P11-12, P11-13, P11-14, P11-15, P11-16, P11-17, P11-18, P11-19, P11-20, P11-24, P11-25, P11-26, P11-27, P11-28, P11-29, P11-30, P11-31, P11-32 and mixtures, and most preferably P11-4, optionally the oligopeptide of the P11-family is associated with a negatively charged polysaccharide or a positively charged polysaccharide.

7. The biomimetic minerizable 3D-printing ink according to ay ene of claim 1, wherein the crystallization trigger is an oligopeptide in which the amino acids at positions 4 and 8 are phenylalanine (F) and the amino acid at position 6 is tryptophan (W) and/or the amino acid residues at both positions 5 and 7 of the peptide are ornithine (O) or glutamic acid (E).

8. The biomimetic minerizable 3D-printing ink according to claim 1, wherein the ink comprises
a) the calcium cation-based compound in an amount ranging from 2 to 30 wt.-%, preferably from 5 to 25 wt.-%, and most preferably from 10 to 25 wt.-%, based on the total weight of the ink, and
b) the carrier material in an amount ranging from 1 to 10 wt.-%, preferably from 1 to 8 wt.-%, and most preferably from 1 to 5 wt.-%, based on the total weight of the ink, and
c) the crystallization trigger in an amount ranging from 0.05 to 3 wt.-%, preferably from 0.05 to 2.8 wt.-%, and most preferably from 0.1 to 2.5 wt.-%, based on the total weight of the ink, and
d) a buffer solution in an amount ranging from 57 to 96.95 wt.-%, preferably from 64.2 to 93.95 wt.-%, and most preferably from 67.5 to 98.9 wt.-%, based on the total weight of the ink.

9. The biomimetic minerizable 3D-printing ink according to claim 1, wherein the calcium cation-based compound being metastable calcium carbonate or metastable calcium phosphate comprises a stabilizer,

10. The biomimetic minerizable 3D-printing ink according to claim 9, wherein the stabilizer is selected from the group comprising magnesium chloride, polyaspartic acid, glutamic acid, polyacrylic acid, phosphates such as L-O-phosphoserine, sodium dihydrogen phosphate and disodium hydrogen phosphate, saccharides, EDTMP, xanthan, polysorbate, citric acid, ehylenediamine, extracts from biogenic samples, double-hydrophilic block copolymers and mixtures thereof.

11. A method for the production of a biomimetic minerizable 3D-printing ink as defined in claim 1, the method comprising the steps of
a) providing a calcium cation-based compound being metastable calcium carbonate or metastable calcium phosphate,
b) providing a carrier material,
c) providing a crystallization trigger, and
d) mixing the calcium cation-based compound being metastable calcium carbonate or metastable calcium phosphate of step a), the carrier material of step b) and the crystallization trigger of step c).

12. A method for the production of a biomineralized 3D-printed article, the method comprising the steps of
a) providing a biomimetic minerizable 3D-printing ink as defined in claim 1,
b) printing the biomimetic minerizable 3D-printing ink into a predetermined form by using a 3D-printer, and
c) hardening the biomimetic minerizable 3D-printing ink at a temperature ranging from 10 to 50° C., preferably from 15 to 45° C., for obtaining the biomineralized 3D-printed article.

13. The method according to claim 12, wherein the hardening in step c) is carried out at
a) a temperature ranging from 15 to 40° C. and most preferably at a temperature ranging from 20 to 40° C., and/or
b) a hardening time ranging from 2 hours to 21 days, preferably from 12 hours to 19 days and most preferably from 24 hours to 18 days, and/or
c) a CO₂ content in the atmosphere from 3 to 6 vol.-%, preferably 4 to 6 vol-% and most preferably from 4.5 to 5.5 vol-%, and/or
d) a humidity of more than 75 vol. %, preferably in the range from 80 to 100 vol. % and most preferably in the range from 85 to 99.5 vol.-%.

14. The method according to claim 12, wherein the method comprises a further step d) of drying the biomineralized 3D-printed article obtained in step c), preferably at a temperature of at least 50° C., more preferably at a temperature ranging from 60 to 100° C. and most preferably from 65 to 90° C., and/or a drying time ranging from 5 hours to 36 hours, preferably from 5 hours to 32 hours and most preferably from 6 hours to 26 hours.

15. A biomineralized 3D-printed article according to claim 12.

16. The biomineralized 3D-printed article according to claim 15, wherein the article is a dental reconstruction material, ceramic substitute, mollusk shell substitute, nacre substitute, dentin substitute, tooth substitute or bone substitute.

17. The method of claim 11, wherein the crystallization trigger is an oligopeptide selected from the group comprising an oligopeptide of the HABP family and an oligopeptide of the P11-family.
